(19) United States
(12) Patent Application Publication
(10) Pub. No.: US 2026/0144758 A1
(43) Pub. Date: May 28, 2026

(54) Hollow Hydroxyapatite Microparticles for Controlled Release of Osteoprotegerin and Their Use in Orthodontic and Bone Regenerative Therapies

(71) Applicant: The Regents of the University of California, Oakland, CA (US)

(72) Inventors: Sunil Kapila, San Francisco, CA (US); Tejal A. Desai, San Francisco, CA (US); Darnell Cuylear, San Francisco, CA (US)

(21) Appl. No.: 19/126,194

(22) PCT Filed: Nov. 6, 2023
(86) PCT No.: PCT/US2023/078824

§ 371 Date: Apr. 30, 2025

Related U.S. Application Data
(60) Provisional application No. 63/423,185, filed on Nov. 7, 2022.

(51) Int. Cl. A61K 9/50; A61K 9/00; A61K 38/18; A61P 19/10

(52) U.S. Cl. CPC A61K 9/501; A61K 9/0053; A61K 9/5089; A61K 38/1875; A61P 19/10

(57) ABSTRACT

Hollow hydroxyapatite microparticles are provided for sustained delivery of an agent that provides osteoprotegerin anti-osteolytic activity for treatment of post-orthodontic relapse and bone disorders. In particular, methods of using such microparticles for post-orthodontic tooth stabilization and bone regeneration and repair are provided.

### CROSS-REFERENCE TO RELATED APPLICATIONS

This application claims the benefit of U.S. Provisional Patent Application No. 63/423,185, filed Nov. 7, 2022, which applications is incorporated herein by reference in its entirety.

### BACKGROUND OF THE INVENTION

The number of orthodontic patients in the U.S. is estimated at about 4.8 million and is increasing annually, especially amongst the adult population. Orthodontic relapse, which entails the rebound of teeth towards their pretreatment positions, is one of the most pressing issues in the field. Due to orthodontic relapse, patients often require repeat treatment to correct their misaligned teeth to decrease the risk of future functional and dental problems including tooth decay, gum disease, and difficulties breathing and/or chewing at substantial cost to patients and reputational and medico-legal risks to providers. Currently, clinicians rely on removeable or bonded retainers to prevent teeth from shifting back to their original positions. However, patient compliance with removable retainers is unpredictable and bonded retainers suffer from long-term failure and increased calculus and plaque deposition. There are currently no therapies available to predictably prevent post-orthodontic relapse. Therefore, there is substantial unmet need for a new innovative approach to improve clinical outcomes for patients, orthodontist, and other dental health providers.

### SUMMARY OF THE INVENTION

Hollow hydroxyapatite microparticles are provided for sustained delivery of an agent that provides osteoprotegerin anti-osteolytic activity for treatment of post-orthodontic relapse and bone disorders. In particular, methods of using such microparticles for post-orthodontic tooth stabilization and bone regeneration and repair are provided.

In one aspect, a microparticle is provided, the microparticle comprising: a) a hollow core; b) a layer of hydroxyapatite encapsulating the hollow core; and c) an agent that provides osteoprotegerin anti-osteolytic activity for bone regeneration or post-orthodontic tooth stabilization, wherein the agent that provides osteoprotegerin is contained within the hollow core.

In certain embodiments, the agent that provides osteoprotegerin activity is osteoprotegerin.

In certain embodiments, the agent that provides osteoprotegerin anti-osteolytic activity is a fusion protein comprising osteoprotegerin. In some embodiments, the fusion protein comprises osteoprotegerin linked to an immunoglobulin constant fragment (Fc).

In certain embodiments, the osteoprotegerin is human osteoprotegerin.

In certain embodiments, the microparticle is a coarse surface hollow microparticle (CM) or a needle surface hollow microparticle (NM).

In certain embodiments, the microparticle has a diameter ranging from about 1.5 μm to about 5.4 μm as measured by transmission electron microscopy (TEM). In some embodiments, the microparticle has a diameter of 2.7 μm±0.3 μm as measured by transmission electron microscopy (TEM).

In certain embodiments, the layer comprising hydroxyapatite has a thickness sufficient to allow sustained release of effective amounts of the OPG from the microparticle for at least 40 days.

In another aspect, a composition comprising a microparticle, described herein, and a pharmaceutically acceptable excipient or carrier is provided. In some embodiments, the carrier is selected from the group consisting of an aqueous solution, a gel, a lotion, a balm, or a paste.

In certain embodiments, the composition comprises an effective dose of the agent that provides osteoprotegerin anti-osteolytic activity to stimulate bone formation and decrease bone resorption.

In certain embodiments, a composition comprising a microparticle, described herein, for use in a method of treating post-orthodontic relapse is provided.

In certain embodiments, a composition comprising the microparticle, described herein, for use in a method of treating a bone disorder is provided.

In another aspect, a kit is provided, the kit comprising a composition comprising a microparticle, described herein, and instructions for using the composition for treating molar relapse or regenerating bone for treatment of a bone disorder.

In another aspect, a method of treating post-orthodontic relapse in a subject in need thereof is provided, the method comprising administering a therapeutically effective amount of a composition comprising a microparticle described herein, to the subject before, after, or during an orthodontic procedure to mitigate or prevent post-orthodontic relapse.

In certain embodiments, the composition is administered submucosally or topically.

In certain embodiments, the composition is administered by intrasulcular or peri-radicular micro-injection.

In another aspect, a method of treating periodontal disease in a subject in need thereof is provided, the method comprising administering a therapeutically effective amount of a composition comprising a microparticle, described herein, to the subject.

In certain embodiments, the composition is administered submucosally or topically.

In certain embodiments, the composition is administered by intrasulcular or peri-radicular micro-injection.

In another aspect, a method of treating a subject having a bone in need of bone regeneration or repair is provided, the method comprising locally administering to the bone a therapeutically effective amount of a composition comprising a microparticle, described herein.

In certain embodiments, the bone has a defect caused by trauma, surgery, a congenital anomaly, or pathological bone loss. In some embodiments, the pathological bone loss is caused by cancer, periodontal disease, osteoporosis, arthritis, or osteonecrosis.

In certain embodiments, the subject is human.

In certain embodiments, the osteoprotegerin is human osteoprotegerin.

In certain embodiments, the composition comprising the microparticle is administered by submucosal, intraperiosteal, or extra-bony injection.

In certain embodiments, multiple therapeutically effective doses of the composition are administered to the subject. In some embodiments, multiple cycles of treatment are administered to the subject for a time period sufficient to effect at least partial regeneration or repair of the bone. In some embodiments, multiple cycles of treatment are administered to the subject for a time period sufficient to effect complete regeneration or repair of the bone.

### BRIEF DESCRIPTION OF THE DRAWINGS

FIG. 1. Schematic of HHAP microparticle synthesis.

FIGS. 2A-2C. Schematics of HHAP microparticle loaded with OPG (FIG. 2A) utilized for (FIG. 2B) post-orthodontic tooth stabilization via microinjection or topical application from retainers, and (FIG. 2C) regenerative therapies for alveolar and long bones to repair defects resulting from disease, developmental defects, surgery and trauma via microinjection or embedded into a scaffold.

FIG. 3. Scanning electron microscopy (SEM), Brunauer-Emmett-Teller (BET) surface area analysis, pore volume, and zeta potential characterization of microparticles.

FIG. 4. Calcium dissolution and ATR FTIR carbonate: phosphate ratio analysis.

FIG. 5. Design and Fabrication of HHAP microparticles: (left) reaction in ice water bath to reduce CaCO₃ aggregation and (right) filtration through 40 μM filter and etching for 5 minutes.

FIG. 6. Adjustment of synthesis parameters for fabrication of HHAP microparticles.

FIG. 7. 50 min. (pH 11) exhibit hollowness and plate-like morphology on surface

FIG. 8. XRD and ATR FTIR confirm the presence of carbonated HAP.

FIG. 9. 50 min. (pH 11) HHAPs exhibit sustained release for 41 days.

FIG. 10. Mineral immaturity index and crystallinity do not change after release in DPBS.

FIG. 11. 50 min. (pH 11) dissolution increases when OPG-Fc is loaded.

FIG. 12. Altering hydrothermal processing times and pH will result in slower release rates by densifying the shell.

FIG. 13. Testing synthesis parameters: Na₂PO₄ pH adjusted to 11 OR 9.14 (pH 9) and process time set at 50 min. OR 30 min.

FIG. 14. pH alters surface topography.

FIG. 15. pH alters surface topography.

FIG. 16. Varying pH effects surface topography and composition.

FIG. 17. Varying pH effects surface topography and composition.

FIG. 18. CM and NM exhibit high loading and controllable release longer than 40 days.

FIG. 19. CM and NM are free of endotoxin contamination.

FIG. 20. OPG-loaded microparticles inhibit osteoclast differentiation.

FIG. 21. NM induces higher osteogenic response in MC3T3-E1 cells.

FIG. 22. NM induces higher osteogenic response in MC3T3-E1 cells.

FIG. 23. NM induces higher osteogenic response in MC3T3-E1 cells.

FIG. 24. Effects are not attributed to mineral quality.

FIG. 25. OPG-Fc loading is not altered with different pH conditions.

FIG. 26. 50 min. (pH 9) release rate is altered, but cumulative is similar

FIG. 27. Calcium dissolution from pH 9 is greater without OPG present.

FIG. 28. In the presence of OPG-Fc, 50 min. (pH 9) dissolution is unaffected.

FIG. 29. Modulation of HHAP bioactivity to alter release kinetics.

FIG. 30. Surface morphology is altered after release by surface bioactivity.

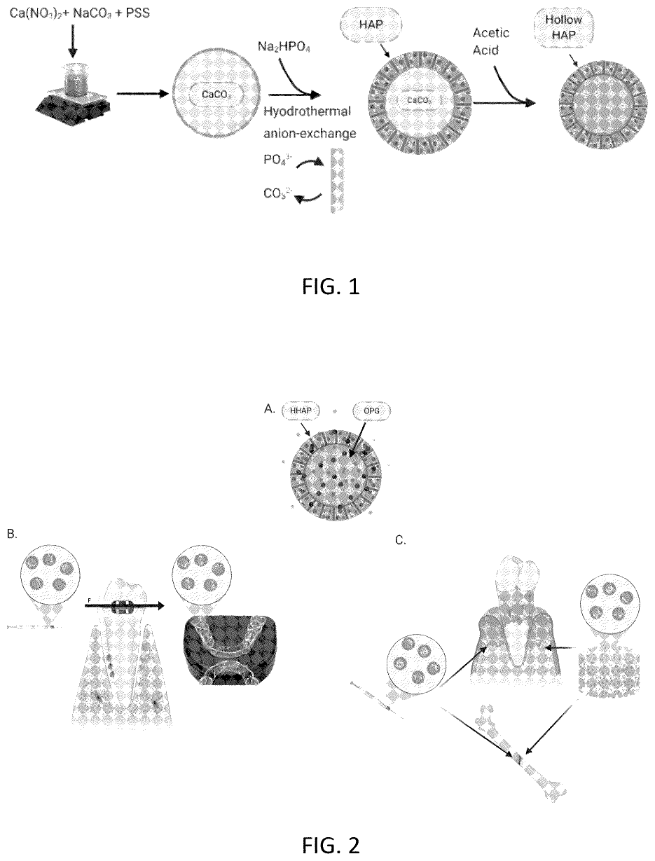

### DETAILED DESCRIPTION OF THE INVENTION

Hydroxyapatite microparticles, methods, and kits are provided for sustained delivery of OPG. In particular, hollow hydroxyapatite microparticles encapsulating an agent that provides osteoprotegerin anti-osteolytic activity are provided for treatment of post-orthodontic relapse and bone disorders. Methods of using such microparticles for post-orthodontic tooth stabilization and bone regeneration and repair are provided.

Before the present compositions, methods, and kits are described, it is to be understood that this invention is not limited to particular methods or compositions described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the microparticle” includes reference to one or more microparticles and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

### Definitions

The term “about,” particularly in reference to a given quantity, is meant to encompass deviations of plus or minus five percent.

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, etc. Preferably, the mammal is human.

“Biocompatible,” as used herein, refers to a property of a material that allows for prolonged contact with a tissue in a subject without causing toxicity or significant damage.

“Substantially” as used herein, may be applied to modify any quantitative representation that could permissibly vary without resulting in a change in the basic function to which it is related.

“Diameter” as used in reference to a shaped structure (e.g., a microparticle) refers to a length that is representative of the overall size of the structure. The length may in general be approximated by the diameter of a circle or sphere that circumscribes the structure.

A “plurality” contains at least 2 members. In certain cases, a plurality may have at least 10, at least 100, at least 1000, at least 10,000, at least 100,000, at least 10⁶, at least 10⁷, at least 10⁸ or at least 10⁹ or more members.

“Substantially purified” generally refers to isolation of a substance (e.g., compound, microparticle, polynucleotide, protein, peptide, antibody, aptamer) such that the substance comprises the majority percent of the sample in which it resides. Typically in a sample, a substantially purified component comprises at least 50%, preferably at least 80%-85%, and more preferably at least 90-95% of the sample. Techniques for purifying substances of interest are well-known in the art and include, for example, ion-exchange chromatography, affinity chromatography, gel filtration, and sedimentation according to density.

As used herein, the terms “treat,” “treatment,” “treating,” and the like, refer to obtaining a desired pharmacologic and/or physiologic effect. The effect may be prophylactic in terms of completely or partially preventing a disease or symptom thereof (e.g., preventing or mitigating post-orthodontic relapse) and/or may be therapeutic in terms of a partial or complete cure for a disease and/or adverse effect attributable to the disease. “Treatment,” as used herein, covers any treatment of a disease in a subject, particularly in a human, and includes: (a) preventing the disease from occurring in a subject which may be predisposed to the disease but has not yet been diagnosed as having it; (b) inhibiting the disease, i.e., arresting its development; and (c) relieving the disease, e.g., causing regression of the disease, e.g., to completely or partially remove symptoms of the disease.

“Therapeutically effective amount” refers to an amount effective, at dosages and for periods of time necessary, to achieve the desired therapeutic result.

“Pharmaceutically acceptable excipient or carrier” refers to an excipient that may optionally be included in the compositions of the invention and that causes no significant adverse toxicological effects to the patient.

“Pharmaceutically acceptable salt” includes, but is not limited to, amino acid salts, salts prepared with inorganic acids, such as chloride, sulfate, phosphate, diphosphate, bromide, and nitrate salts, or salts prepared from the corresponding inorganic acid form of any of the preceding, e.g., hydrochloride, etc., or salts prepared with an organic acid, such as malate, maleate, fumarate, tartrate, succinate, ethylsuccinate, citrate, acetate, lactate, methanesulfonate, benzoate, ascorbate, para-toluenesulfonate, palmoate, salicylate and stearate, as well as estolate, gluceptate and lactobionate salts. Similarly, salts containing pharmaceutically acceptable cations include, but are not limited to, sodium, potassium, calcium, aluminum, lithium, and ammonium (including substituted ammonium).

The term “bone disorder” refers to any disease or condition that produces a defect in a bone. The bone defect may be caused by trauma, surgery, a congenital anomaly, or pathological bone loss. A bone disorder may include, but is not limited to, cancer, periodontal disease, osteoporosis, arthritis, and osteonecrosis.

The terms “polypeptide,” “peptide” and “protein” are used interchangeably herein to refer to a polymer of amino acid residues. The terms also apply to amino acid polymers in which one or more amino acid residues are artificial chemical mimetics of a corresponding naturally occurring amino acid, as well as to naturally occurring amino acid polymers and non-naturally occurring amino acid polymers. Both full-length proteins and fragments thereof are encompassed by the definition. The terms also include postexpression modifications of the polypeptide, for example, phosphorylation, glycosylation, acetylation, hydroxylation, oxidation, and the like.

The terms “polynucleotide,” “oligonucleotide,” “nucleic acid” and “nucleic acid molecule” are used herein to include a polymeric form of nucleotides of any length, either ribonucleotides or deoxyribonucleotides. This term refers only to the primary structure of the molecule. Thus, the term includes triple-, double-and single-stranded DNA, as well as triple-, double-and single-stranded RNA. It also includes modifications, such as by methylation and/or by capping, and unmodified forms of the polynucleotide. More particularly, the terms “polynucleotide,” “oligonucleotide,” “nucleic acid” and “nucleic acid molecule” include polydeoxyribonucleotides (containing 2-deoxy-D-ribose), polyribonucleotides (containing D-ribose), and any other type of polynucleotide which is an N-or C-glycoside of a purine or pyrimidine base. There is no intended distinction in length between the terms “polynucleotide,” “oligonucleotide,” “nucleic acid” and “nucleic acid molecule,” and these terms are used interchangeably.

By “isolated” is meant, when referring to a protein, polypeptide, or peptide, that the indicated molecule is separate and discrete from the whole organism with which the molecule is found in nature or is present in the substantial absence of other biological macro molecules of the same type. The term “isolated” with respect to a polynucleotide is a nucleic acid molecule devoid, in whole or part, of sequences normally associated with it in nature; or a sequence, as it exists in nature, but having heterologous sequences in association therewith; or a molecule disassociated from the chromosome.

As used herein, the terms “increase”, “increasing”, “enhance”, and “enhancing” (and grammatical variations thereof) describes unless the context indicates otherwise a detectable elevation of a reference value. An increase can comprise an elevation of at least about 5%, 10%, 15%, 20%, 25%, 30%, 35%, 40%, 45%, 50%, 55%, 60%, 65%, 70%, 75%, 80%, 85%, 90%, 95%, 100%, 150%, 200%, 300%, 400%, 500% or more such as compared to another measurable property or quantity (e.g., a control value).

An osteoprotegerin polynucleotide, nucleic acid, oligonucleotide, protein, polypeptide, or peptide refers to a molecule derived from any source. The molecule need not be physically derived from an organism, but may be synthetically or recombinantly produced. A number of osteoprotegerin nucleic acid and protein sequences are known. Representative osteoprotegerin sequences are listed in the National Center for Biotechnology Information (NCBI) database. See, for example, NCBI entries: Accession Nos. NM_033012, NM_003701, NM_008764, NM_002546, NM_001411506, NM_008764, NM_012870, NM_001098056, NM_001092818, NM_001349196, NC_000081, NM_001033641, NP_002537, NP_143026, NP_003692, NP_001398435, NP_037002, NP_001091525, NP_001239168, and NP_001336125; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, or a biologically active fragment of osteoprotegerin, which retains osteoprotegerin anti-osteolytic activity, can be encapsulated within a HHAP microparticle or used to construct a fusion protein (e.g., osteoprotegerin-Fc) that is encapsulated in a HHAP microparticle, as described herein. Additional, examples of osteoprotegerin genes and protein sequences can be found in public gene databases such as NCBI, Uniprot and other public genomic and protein sequence databases identifiable to a person skilled in the art. The osteoprotegerin may be a wild-type human or mammalian homolog or modified or mutated.

As used herein, “reference sequence” is a defined sequence used as a basis for sequence comparison. A reference sequence may be a subset or the entirety of a specified sequence; for example, as a segment of a full-length protein or protein fragment. A reference sequence can comprise, for example, a sequence identifiable in a database such as GenBank and UniProt and others identifiable to those skilled in the art.

It will be apparent to one of ordinary skill in the art that various changes and modifications can be made without departing from the spirit or scope of the invention.

### Microparticles

Hollow hydroxyapatite (HHAP) microparticles are provided for sustained delivery of an agent that provides osteoprotegerin anti-osteolytic activity for stimulating bone regeneration. Such microparticles may be used to repair bone defects caused by trauma, surgery, a congenital anomaly, or pathological bone loss and may be used to treat various bone disorders including, but not limited to post-orthodontic relapse, cancer, periodontal disease, osteoporosis, arthritis, and osteonecrosis.

A HHAP microparticle comprises a) a hollow core; b) a layer of hydroxyapatite encapsulating the hollow core; and c) an agent that provides osteoprotegerin anti-osteolytic activity for bone regeneration or post-orthodontic tooth stabilization, wherein the agent that provides osteoprotegerin is contained within the hollow core. In certain embodiments, the microparticle is a coarse surface hollow microparticle (CM) or a needle surface hollow microparticle (NM).

In some embodiments, the microparticle has a diameter ranging from about 1.5 μm to about 5.4 μm, including any diameter within this range, such as 1.5 μm, 1.75 μm, 2.0 μm, 2.25 μm, 2.5 μm, 2.75 μm, 3.0 μm, 3.25 μm, 3.5 μm, 3.75 μm, 4.0 μm, 4.25 μm, 4.5 μm, 4.75 μm, 5.0 μm, 5.25 μm, or 5.4 μm, as measured by transmission electron microscopy (TEM). In some embodiments, the microparticle has a diameter of about 1.5 nm or more, e.g., about 2.0 nm or more, about 2.5 nm or more, including about 3.0 nm or more, and may have a diameter of about 5.0 nm or less, e.g., about 4.5 nm or less, about 4.0 nm or less, about 3.5 nm or less, about 3.00 nm or less, including about 2.75 nm or less. In some embodiments, the microparticle has a diameter of 2.7 μm±0.3 μm as measured by TEM.

In some embodiments, the microparticle is loaded with an agent that provides osteoprotegerin anti-osteolytic activity such as an osteoprotegerin protein or a variant or biologically active fragment thereof having anti-osteolytic activity, or a fusion protein comprising osteoprotegerin linked to an immunoglobulin constant fragment (Fc). In some embodiments, the Fc is a human lgG1 Fc.

Osteoprotegerin nucleic acid and protein sequences may be derived from any source. A number of osteoprotegerin nucleic acid and protein sequences are known. Representative osteoprotegerin sequences are listed in the National Center for Biotechnology Information (NCBI) database. See, for example, NCBI entries: Accession Nos. NM_033012, NM_003701, NM_008764, NM_002546, NM_001411506, NM_008764, NM_012870, NM_001098056, NM_001092818, NM_001349196, NC_000081, NM_001033641, NP_002537, NP_143026, NP_003692, NP_001398435, NP_037002, NP_001091525, NP_001239168, and NP_001336125; all of which sequences (as entered by the date of filing of this application) are herein incorporated by reference. Any of these sequences or a variant thereof comprising a sequence having at least about 80-100% sequence identity thereto, including any percent identity within this range, such as 81, 82, 83, 84, 85, 86, 87, 88, 89, 90, 91, 92, 93, 94, 95, 96, 97, 98, or 99% sequence identity thereto, or a biologically active fragment of osteoprotegerin, which retains osteoprotegerin anti-osteolytic activity, can be encapsulated within a HHAP microparticle or used to construct a fusion protein (e.g., osteoprotegerin-Fc) that is encapsulated in a HHAP microparticle, as described herein. Additional, examples of osteoprotegerin genes and protein sequences can be found in public gene databases such as NCBI, Uniprot and other public genomic and protein sequence databases identifiable to a person skilled in the art. The osteoprotegerin may be a wild-type human or mammalian homolog or modified or mutated.

In some embodiments, osteoprotegerin or an osteoprotegerin-containing fusion protein is generated using recombinant techniques. One of skill in the art can readily determine nucleotide sequences that encode the desired osteoprotegerin protein or fusion protein using standard methodology and the teachings herein. Oligonucleotide probes can be devised based on the known sequences and used to probe genomic or cDNA libraries. The sequences can then be further isolated using standard techniques and, e.g., restriction enzymes employed to truncate the gene at desired portions of the full-length sequence. Similarly, sequences of interest can be isolated directly from cells and tissues containing the same, using known techniques, such as phenol extraction and the sequence further manipulated to produce the desired truncations. See, e.g., Sambrook et al., supra, for a description of techniques used to obtain and isolate DNA.

The sequences encoding an osteoprotegerin protein or fusion protein can also be produced synthetically, for example, based on the known sequences. The nucleotide sequence can be designed with the appropriate codons for the particular amino acid sequence desired. The complete sequence is generally assembled from overlapping oligonucleotides prepared by standard methods and assembled into a complete coding sequence. See, e.g., Edge (1981) Nature 292:756; Nambair et al. (1984) Science 223:1299; Jay et al. (1984) J. Biol. Chem. 259:6311; Stemmer et al. (1995) Gene 164:49-53.

Recombinant techniques are readily used to clone sequences encoding proteins or fusion proteins that can then be mutagenized in vitro by the replacement of the appropriate base pair(s) to result in the codon for the desired amino acid. Such a change can include as little as one base pair, effecting a change in a single amino acid, or can encompass several base pair changes. Alternatively, the mutations can be effected using a mismatched primer that hybridizes to the parent nucleotide sequence (generally cDNA corresponding to the RNA sequence), at a temperature below the melting temperature of the mismatched duplex. The primer can be made specific by keeping primer length and base composition within relatively narrow limits and by keeping the mutant base centrally located. See, e.g., Innis et al, (1990) PCR Applications: Protocols for Functional Genomics; Zoller and Smith, Methods Enzymol. (1983) 100:468. Primer extension is effected using DNA polymerase, the product cloned and clones containing the mutated DNA, derived by segregation of the primer extended strand, selected. Selection can be accomplished using the mutant primer as a hybridization probe. The technique is also applicable for generating multiple point mutations. See, e.g., Dalbie-McFarland et al. Proc. Natl. Acad. Sci USA (1982) 79:6409.

Once coding sequences have been isolated and/or synthesized, they can be cloned into any suitable vector or replicon for expression. As will be apparent from the teachings herein, a wide variety of vectors encoding modified proteins can be generated by creating expression constructs which operably link, in various combinations, polynucleotides encoding proteins having deletions or mutations therein.

Numerous cloning vectors are known to those of skill in the art, and the selection of an appropriate cloning vector is a matter of choice. Examples of recombinant DNA vectors for cloning and host cells which they can transform include the bacteriophage λ (E. coli), pBR322 (E. coli), pACYC177 (E. coli), pKT230 (gram-negative bacteria), pGV1106 (gram-negative bacteria), pLAFR1 (gram-negative bacteria), pME290 (non-E. coli gram-negative bacteria), pHV14 (E. coli and Bacillus subtilis), pBD9 (Bacillus), plJ61 (Streptomyces), pUC6 (Streptomyces), Ylp5 (Saccharomyces), YCp19 (Saccharomyces) and bovine papilloma virus (mammalian cells). See, generally, DNA Cloning: Vols. I & II, supra; Sambrook et al., supra; B. Perbal, supra.

Insect cell expression systems, such as baculovirus systems, can also be used and are known to those of skill in the art and described in, e.g., Summers and Smith, Texas Agricultural Experiment Station Bulletin No. 1555(1987). Materials and methods for baculovirus/insect cell expression systems are commercially available in kit form from, inter alia, Invitrogen, San Diego CA (“MaxBac” kit).

Plant expression systems can also be used to produce an osteoprotegerin protein or fusion protein described herein. Generally, such systems use virus-based vectors to transfect plant cells with heterologous genes. For a description of such systems, see, e.g., Porta et al., Mol. Biotech. (1996) 5:209-221; and Hackland et al., Arch. Virol. (1994) 139:1-22.

Viral systems, such as a vaccinia-based infection/transfection system, as described in Tomei et al., J. Virol. (1993) 67:4017-4026 and Selby et al., J. Gen. Virol. (1993) 74:1103-1113, will also find use with the present invention. In this system, cells are first transfected in vitro with a vaccinia virus recombinant that encodes the bacteriophage T7 RNA polymerase. This polymerase displays exquisite specificity in that it only transcribes templates bearing T7 promoters. Following infection, cells are transfected with the DNA of interest, driven by a T7 promoter. The polymerase expressed in the cytoplasm from the vaccinia virus recombinant transcribes the transfected DNA into RNA that is then translated into protein by the host translational machinery. The method provides for high level, transient, cytoplasmic production of large quantities of RNA and its translation product(s).

The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. With the present invention, both the naturally occurring signal peptides or heterologous sequences can be used. Leader sequences can be removed by the host in post-translational processing. See, e.g., U.S. Pat. Nos. 4,431,739; 4,425,437; 4,338,397. Such sequences include, but are not limited to, the TPA leader, as well as the honeybee mellitin signal sequence.

Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector. Alternatively, the coding sequence can be cloned directly into an expression vector that already contains the control sequences and an appropriate restriction site.

In some cases, it may be necessary to modify the coding sequence so that it may be attached to the control sequences with the appropriate orientation; i.e., to maintain the proper reading frame. Mutants or analogs may be prepared by the deletion of a portion of the sequence encoding the protein, by insertion of a sequence, and/or by substitution of one or more nucleotides within the sequence. Techniques for modifying nucleotide sequences, such as site-directed mutagenesis, are well known to those skilled in the art. See, e.g., Sambrook et al., supra; DNA Cloning, Vols. I and II, supra; Nucleic Acid Hybridization, supra.

The expression vector is then used to transform an appropriate host cell. A number of mammalian cell lines are known in the art and include immortalized cell lines available from the American Type Culture Collection (ATCC), such as, but not limited to, Chinese hamster ovary (CHO) cells, Hela cells, baby hamster kidney (BHK) cells, monkey kidney cells (COS), human hepatocellular carcinoma cells (e.g., Hep G2), Vero293 cells, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni.

Depending on the expression system and host selected, the osteoprotegerin protein or fusion protein are produced by growing host cells transformed by an expression vector described above under conditions whereby the protein of interest is expressed. The selection of the appropriate growth conditions is within the skill of the art.

In one embodiment, the transformed cells secrete the osteoprotegerin protein or fusion protein product into the surrounding media. Certain regulatory sequences can be included in the vector to enhance secretion of the protein product, for example using a tissue plasminogen activator (TPA) leader sequence, an interferon (γ or α) signal sequence or other signal peptide sequences from known secretory proteins. The secreted osteoprotegerin protein or fusion protein product can then be isolated by various techniques described herein, for example, using standard purification techniques such as but not limited to, hydroxyapatite resins, column chromatography, ion-exchange chromatography, size-exclusion chromatography, electrophoresis, HPLC, immunoadsorbent techniques, affinity chromatography, immunoprecipitation, and the like.

Alternatively, the transformed cells are disrupted, using chemical, physical or mechanical means, which lyse the cells yet keep the recombinant proteins substantially intact. Intracellular proteins can also be obtained by removing components from the cell wall or membrane, e.g., by the use of detergents or organic solvents, such that leakage of the proteins occurs. Such methods are known to those of skill in the art and are described in, e.g., Protein Purification Applications: A Practical Approach, (Simon Roe, Ed., 2001).

For example, methods of disrupting cells for use with the present invention include but are not limited to: sonication or ultrasonication; agitation; liquid or solid extrusion; heat treatment; freeze-thaw; desiccation; explosive decompression; osmotic shock; treatment with lytic enzymes including proteases such as trypsin, neuraminidase and lysozyme; alkali treatment; and the use of detergents and solvents such as bile salts, sodium dodecylsulphate, Triton, NP40 and CHAPS. The particular technique used to disrupt the cells is largely a matter of choice and will depend on the cell type in which the osteoprotegerin protein or fusion protein is expressed, culture conditions and any pre-treatment used.

Following disruption of the cells, cellular debris is removed, generally by centrifugation, and the intracellularly produced proteins are further purified, using standard purification techniques such as but not limited to, column chromatography, ion-exchange chromatography, size-exclusion chromatography, electrophoresis, HPLC, immunoadsorbent techniques, affinity chromatography, immunoprecipitation, and the like.

For example, one method for obtaining the intracellular osteoprotegerin protein or fusion protein involves affinity purification, such as by immunoaffinity chromatography using antibodies (e.g., previously generated antibodies), or by lectin affinity chromatography. Particularly preferred lectin resins are those that recognize mannose moieties such as but not limited to resins derived from Galanthus nivalis agglutinin (GNA), Lens culinaris agglutinin (LCA or lentil lectin), Pisum sativum agglutinin (PSA or pea lectin), Narcissus pseudonarcissus agglutinin (NPA) and Allium ursinum agglutinin (AUA). The choice of a suitable affinity resin is within the skill in the art. After affinity purification, the osteoprotegerin protein or fusion protein can be further purified using conventional techniques well known in the art, such as by any of the techniques described above.

The osteoprotegerin protein or fusion protein can be conveniently synthesized chemically, for example by any of several techniques that are known to those skilled in the peptide art. See, e.g., Fmoc Solid Phase Peptide Synthesis: A Practical Approach (W. C. Chan and Peter D. White eds., Oxford University Press, 1^{st} edition, 2000); N. Leo Benoiton, Chemistry of Peptide Synthesis (CRC Press; 1^{st} edition, 2005); Peptide Synthesis and Applications (Methods in Molecular Biology, John Howl ed., Humana Press, 1^{st} ed., 2005); and Pharmaceutical Formulation Development of Peptides and Proteins (The Taylor & Francis Series in Pharmaceutical Sciences, Lars Hovgaard, Sven Frokjaer, and Marco van de Weert eds., CRC Press; 1^{st} edition, 1999); herein incorporated by reference.

In general, these methods employ the sequential addition of one or more amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions that allow for the formation of an amide linkage. The protecting group is then removed from the newly added amino acid residue and the next amino acid (suitably protected) is then added, and so forth. After the desired amino acids have been linked in the proper sequence, any remaining protecting groups (and any solid support, if solid phase synthesis techniques are used) are removed sequentially or concurrently, to render the final osteoprotegerin protein or fusion protein. By simple modification of this general procedure, it is possible to add more than one amino acid at a time to a growing chain, for example, by coupling (under conditions which do not racemize chiral centers) a protected tripeptide with a properly protected dipeptide to form, after deprotection, a pentapeptide. See, e.g., J. M. Stewart and J. D. Young, Solid Phase Peptide Synthesis (Pierce Chemical Co., Rockford, IL 1984) and G. Barany and R. B. Merrifield, The Peptides: Analysis, Synthesis, Biology, editors E. Gross and J. Meienhofer, Vol. 2, (Academic Press, New York, 1980), pp. 3-254, for solid phase peptide synthesis techniques; and M. Bodansky, Principles of Peptide Synthesis, (Springer-Verlag, Berlin 1984) and E. Gross and J. Meienhofer, Eds., The Peptides: Analysis, Synthesis, Biology, Vol. 1, for classical solution synthesis. These methods are typically used for relatively small polypeptides, i.e., up to about 50-100 amino acids in length, but are also applicable to larger polypeptides.

Typical protecting groups include t-butyloxycarbonyl (Boc), 9-fluorenylmethoxycarbonyl (Fmoc) benzyloxycarbonyl (Cbz); p-toluenesulfonyl (Tx); 2,4-dinitrophenyl; benzyl (Bzl); biphenylisopropyloxycarboxy-carbonyl, t-amyloxycarbonyl, isobornyloxycarbonyl, 0-bromobenzyloxycarbonyl, cyclohexyl, isopropyl, acetyl, o-nitrophenylsulfonyl and the like.

Typical solid supports are cross-linked polymeric supports. These can include divinylbenzene cross-linked-styrene-based polymers, for example, divinylbenzene-hydroxymethylstyrene copolymers, divinylbenzene-chloromethylstyrene copolymers and divinylbenzene-benzhydrylaminopolystyrene copolymers.

The osteoprotegerin protein or fusion protein can also be chemically prepared by other methods such as by the method of simultaneous multiple peptide synthesis. See, e.g., Houghten Proc. Natl. Acad. Sci. USA (1985) 82:5131-5135; U.S. Pat. No. 4,631,211.

### Pharmaceutical Compositions

HHAP microparticles encapsulating an agent that provides osteoprotegerin anti-osteolytic activity (e.g., an osteoprotegerin protein or an osteoprotegerin-containing fusion protein) can be formulated into pharmaceutical compositions optionally comprising one or more pharmaceutically acceptable excipients. Exemplary excipients include, without limitation, carbohydrates, inorganic salts, antimicrobial agents, antioxidants, surfactants, buffers, acids, bases, and combinations thereof. Excipients suitable for injectable compositions include water, alcohols, polyols, glycerin, vegetable oils, phospholipids, and surfactants. A carbohydrate such as a sugar, a derivatized sugar such as an alditol, aldonic acid, an esterified sugar, and/or a sugar polymer may be present as an excipient. Specific carbohydrate excipients include, for example: monosaccharides, such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol, sorbitol (glucitol), pyranosyl sorbitol, myoinositol, and the like. The excipient can also include an inorganic salt or buffer such as citric acid, sodium chloride, potassium chloride, sodium sulfate, potassium nitrate, sodium phosphate monobasic, sodium phosphate dibasic, and combinations thereof.

A composition can also include an antimicrobial agent for preventing or deterring microbial growth. Nonlimiting examples of antimicrobial agents suitable for the present invention include benzalkonium chloride, benzethonium chloride, benzyl alcohol, cetylpyridinium chloride, chlorobutanol, phenol, phenylethyl alcohol, phenylmercuric nitrate, thimersol, and combinations thereof.

An antioxidant can be present in the composition as well. Antioxidants are used to prevent oxidation, thereby preventing the deterioration of the microparticle or other components of the preparation. Suitable antioxidants for use in the present invention include, for example, ascorbyl palmitate, butylated hydroxyanisole, butylated hydroxytoluene, hypophosphorous acid, monothioglycerol, propyl gallate, sodium bisulfite, sodium formaldehyde sulfoxylate, sodium metabisulfite, and combinations thereof.

A surfactant can be present as an excipient. Exemplary surfactants include: polysorbates, such as “Tween 20” and “Tween 80,” and pluronics such as F68 and F88 (BASF, Mount Olive, New Jersey); sorbitan esters; lipids, such as phospholipids such as lecithin and other phosphatidylcholines, phosphatidylethanolamines (although preferably not in liposomal form), fatty acids and fatty esters; steroids, such as cholesterol; chelating agents, such as EDTA; and zinc and other such suitable cations.

Acids or bases can be present as an excipient in the composition. Nonlimiting examples of acids that can be used include those acids selected from the group consisting of hydrochloric acid, acetic acid, phosphoric acid, citric acid, malic acid, lactic acid, formic acid, trichloroacetic acid, nitric acid, perchloric acid, phosphoric acid, sulfuric acid, fumaric acid, and combinations thereof. Examples of suitable bases include, without limitation, bases selected from the group consisting of sodium hydroxide, sodium acetate, ammonium hydroxide, potassium hydroxide, ammonium acetate, potassium acetate, sodium phosphate, potassium phosphate, sodium citrate, sodium formate, sodium sulfate, potassium sulfate, potassium fumerate, and combinations thereof.

The amount of any individual excipient in the composition will vary depending on the nature and function of the excipient and particular needs of the composition. Typically, the optimal amount of any individual excipient is determined through routine experimentation, i.e., by preparing compositions containing varying amounts of the excipient (ranging from low to high), examining the stability and other parameters, and then determining the range at which optimal performance is attained with no significant adverse effects. Generally, however, the excipient(s) will be present in the composition in an amount of about 1% to about 99% by weight, preferably from about 5% to about 98% by weight, more preferably from about 15 to about 95% by weight of the excipient, with concentrations less than 30% by weight most preferred. These foregoing pharmaceutical excipients along with other excipients are described in “Remington: The Science & Practice of Pharmacy”, 19th ed., Williams & Williams, (1995), the “Physician's Desk Reference”, 52nd ed., Medical Economics, Montvale, NJ (1998), and Kibbe, A.H., Handbook of Pharmaceutical Excipients, 3rd Edition, American Pharmaceutical Association, Washington, D.C., 2000.

The compositions encompass all types of formulations and in particular those that are suited for injection, e.g., powders or lyophilates that can be reconstituted with a solvent prior to use, as well as ready for injection solutions or suspensions, dry insoluble compositions for combination with a vehicle prior to use, and emulsions and liquid concentrates for dilution prior to administration. Examples of suitable diluents for reconstituting solid compositions prior to injection include bacteriostatic water for injection, dextrose 5% in water, phosphate buffered saline, Ringer's solution, saline, sterile water, deionized water, and combinations thereof. With respect to liquid pharmaceutical compositions, solutions and suspensions are envisioned. Additional preferred compositions include those for topical or localized delivery.

The pharmaceutical preparations herein can also be housed in a syringe, an implantation device, or the like, depending upon the intended mode of delivery and use. Preferably, the compositions comprising microparticles described herein are in unit dosage form, meaning an amount of microparticles appropriate for a single dose, in a premeasured or pre-packaged form.

The compositions herein may optionally include one or more additional agents, such as anti-inflammatory, immunosuppressive, analgesic, or chemotherapeutic drugs or other medications used to treat a subject for a bone disorder. For example, compounded preparations may include microparticles and one or more drugs for treating a condition or disease. Alternatively, such agents can be contained in a separate composition from the composition comprising the microparticles and co-administered concurrently, before, or after the composition comprising the microparticles.

### Administration

At least one therapeutically effective cycle of treatment with HHAP microparticles encapsulating an agent that provides osteoprotegerin anti-osteolytic activity (e.g., an osteoprotegerin protein or an osteoprotegerin-containing fusion protein) will be administered to a subject for treatment of a bone disorder, which may include any disease or condition that produces a defect in a bone caused, for example, by trauma, surgery, an orthodontic procedure, a congenital anomaly, or pathological bone loss. Bone disorders include, but are not limited to, post-orthodontic relapse, cancer, periodontal disease, osteoporosis, arthritis, and osteonecrosis.

By “therapeutically effective dose or amount” of HHAP microparticles encapsulating an agent that provides osteoprotegerin anti-osteolytic activity (e.g., an osteoprotegerin protein or an osteoprotegerin-containing fusion protein) is intended an amount that, when administered, as described herein, brings about a positive therapeutic response, such as improved recovery from a bone disorder. Improved recovery may include bone regeneration and at least partial or complete repair of a bone defect. In the case of post-orthodontic relapse, a subject may be administered a therapeutically effective dose or amount of HHAP microparticles encapsulating an agent that provides osteoprotegerin anti-osteolytic activity before, during, or after an orthodontic procedure to provide post-orthodontic tooth stabilization and mitigate or prevent post-orthodontic relapse.

In certain embodiments, multiple therapeutically effective doses of compositions comprising HHAP microparticles encapsulating an agent that provides osteoprotegerin anti-osteolytic activity, and/or one or more other therapeutic agents, such as other drugs for treating a bone disorder, or other medications will be administered.

A therapeutically effective amount of a composition comprising microparticles is administered to a subject; that is, an amount that supplies an effective amount of the agent that provides osteoprotegerin anti-osteolytic activity (e.g., an osteoprotegerin protein or an osteoprotegerin-containing fusion protein) encapsulated therein (e.g., contained in the hollow core of the microparticle) for a period of time sufficient to stimulate bone regeneration. A therapeutically effective amount of the microparticles may be administered in more than one administration if needed. The therapeutically effective amount of the microparticles needed for an individual may vary according to factors such as the degree of uptake of the microparticles into a tissue of interest, the particular bone disorder for which an individual is undergoing treatment, and the age, sex, and weight of the individual. Optimization of such factors is within the level of skill in the art.

The compositions comprising microparticles are typically, though not necessarily, administered locally. For example, compositions comprising microparticles may be administered in proximity to a bone in need of regeneration or repair. Administration may be, for example, by injection, surgical implantation, perfusion through a regional catheter, or direct injection. When administering the microparticles by injection, the administration may be by continuous infusion or by single or multiple boluses.

In the case of treatment of post-orthodontic relapse or periodontal disease, micropartcles may be administered submucosally or topically. In certain embodiments, the microparticles are administered by intrasulcular or peri-radicular micro-injection around sites where post-orthodontic tooth stabilization or orthodontic anchorage is needed. In some embodiments, a pharmaceutical composition comprising the microparticles is applied to a short-term retainer worn by a patient for delivery of the OPG.

For bone regenerative therapy including for rapid stabilization and bone repair following, e.g., osteotomies, pathologic, traumatic, or surgical bone loss, or for non-unions, the microparticles may be administered by submucosal, intraperiosteal, or extra-bony injection following elevation of soft tissues to deposit the microparticles.

In some embodiments, the administration method may include implanting (e.g., injecting or surgically implanting) the microparticles in proximity to the bone in need of regeneration or repair in a patient. A medical practitioner may locate the site for implantation of microparticles (e.g., in proximity to a bone defect to be treated), for example, by medical imaging (e.g., ultrasound, radiography, or MRI). In some embodiments, a contrast agent is included in the composition comprising the microparticles to allow confirmation of the location of the microparticles by medical imaging after implantation. In some embodiments, the contrast agent is a microbubble (e.g., for use in ultrasound) or a radiopaque contrast agent (e.g., for use in radiography). The contrast agent may be contained in the same composition as the microparticles or in a different composition and used prior to or after implantation of the microparticles.

The pharmaceutical preparation can be in the form of a liquid solution or suspension immediately prior to administration, but may also take another form such as a syrup, cream, ointment, tablet, capsule, powder, gel, matrix, suppository, or the like. The pharmaceutical compositions comprising microparticles may be administered in accordance with any medically acceptable method known in the art.

### Kits

Kits comprising HHAP microparticles encapsulating an agent that provides osteoprotegerin anti-osteolytic activity (e.g., an osteoprotegerin protein or an osteoprotegerin-containing fusion protein) are also provided. The kit may also include a packaging that includes a compartment, e.g., a sterile compartment, for holding the microparticles. Compositions comprising microparticles can be suspended in a liquid or can be lyophilized.

Suitable containers for the compositions include, for example, bottles, vials, syringes, and test tubes. Containers can be formed from a variety of materials, including glass or plastic. A container may have a sterile access port (for example, the container may be an intravenous solution bag or a vial having a stopper pierceable by a hypodermic injection needle). The kit can further comprise a container comprising a pharmaceutically-acceptable buffer, such as phosphate-buffered saline, Ringer's solution, or dextrose solution. It can also contain other materials useful to the end-user, including other pharmaceutically acceptable formulating solutions such as buffers, diluents, filters, needles, and syringes or other delivery device. The kit may also provide a delivery device pre-filled with the microparticles.

In addition to the above components, the subject kits may further include (in certain embodiments) instructions for practicing the subject methods (i.e., instructions for treating a bone disorder with the microparticles, as described herein). These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), DVD, Blu-ray, flash drive, and the like, on which the information has been recorded. Yet another form of these instructions that may be present is a website address which may be used via the internet to access the information at a removed site.

### Utility

The HHAP microparticles will be useful for providing sustained delivery of osteoprotegerin for treating a variety of bone disorders, including any disease or condition that produces a defect in a bone. For example, such microparticles can be used to stimulate bone regeneration and repair bone defects such as caused by trauma, surgery, a congenital anomaly, or pathological bone loss. In particular, microparticles can be used to mitigate or prevent post-orthodontic relapse and treat pathological bone loss caused by diseases such as, but not limited to cancer, periodontal disease, osteoporosis, arthritis, and osteonecrosis.

### Examples of Non-limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure numbered 1-30 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below.
1. A microparticle comprising:
   a) a hollow core;
   b) a layer of hydroxyapatite encapsulating the hollow core; and
   c) an agent that provides osteoprotegerin anti-osteolytic activity for bone regeneration or post-orthodontic tooth stabilization, wherein the agent that provides osteoprotegerin is contained within the hollow core.
2. The microparticle of aspect 1, wherein the agent that provides osteoprotegerin anti-osteolytic activity is osteoprotegerin.
3. The microparticle of aspect 1, wherein the agent that provides osteoprotegerin anti-osteolytic activity is a fusion protein comprising osteoprotegerin.
4. The microparticle of aspect 3, wherein the fusion protein comprises osteoprotegerin linked to an immunoglobulin constant fragment (Fc).
5. The microparticle of any one of aspects 1-4, wherein the osteoprotegerin is human osteoprotegerin.
6. The microparticle of any one of aspects 1-5, wherein the microparticle is a coarse surface hollow microparticle (CM) or a needle surface hollow microparticle (NM).
7. The microparticle of any one of aspects 1-6, wherein the microparticle has a diameter ranging from about 1.5 μm to about 5.4 μm as measured by transmission electron microscopy (TEM).
8. The microparticle of aspect 7 wherein the microparticle has a diameter of 2.7 μm±0.3 μm as measured by transmission electron microscopy (TEM).
9. The microparticle of any one of aspects 1-8, wherein the layer comprising hydroxyapatite has a thickness sufficient to allow sustained release of effective amounts of the OPG from the microparticle for at least 40 days.
10. A composition comprising the microparticle of any one of aspects 1-9 and a pharmaceutically acceptable excipient or carrier.
11. The composition of aspect 10, wherein the composition comprises an effective dose of the agent that provides osteoprotegerin anti-osteolytic activity to stimulate bone formation and decrease bone resorption.
12, A composition comprising the microparticle of any one of aspects 1-9 for use in a method of treating post-orthodontic relapse.
13, A composition comprising the microparticle of any one of aspects 1-9 for use in a method of treating a bone disorder.
14. The composition of any one of aspects 10-13, wherein the carrier is selected from the group consisting of an aqueous solution, a gel, a lotion, a balm, or a paste.
15. A kit comprising the composition of any one of aspects 10-14 and instructions for using the composition for treating molar relapse or regenerating bone for treatment of a bone disorder.
16. A method of treating post-orthodontic relapse in a subject in need thereof, the method comprising administering a therapeutically effective amount of the composition of any one of aspects 10-14 to the subject before, after, or during an orthodontic procedure to mitigate or prevent post-orthodontic relapse.
17. The method of aspect 16, wherein the composition is administered submucosally or topically.
18. The method of aspect 16, wherein the composition is administered by intrasulcular or peri-radicular micro-injection.
19. A method of treating periodontal disease in a subject in need thereof, the method comprising administering a therapeutically effective amount of the composition of any one of aspects 10-14 to the subject.
20. The method of aspect 19, wherein the composition is administered submucosally or topically.
21. The method of aspect 19, wherein the composition is administered by intrasulcular or peri-radicular micro-injection.
22. A method of treating a subject having a bone in need of bone regeneration or repair, the method comprising locally administering to the bone a therapeutically effective amount of the composition of any one of aspects 10-14.
23. The method of aspect 22, wherein the bone has a defect caused by trauma, surgery, a congenital anomaly, or pathological bone loss.
24. The method of aspect 23, wherein the pathological bone loss is caused by cancer, periodontal disease, osteoporosis, arthritis, or osteonecrosis.
25. The method of any one of aspects 22-24, wherein the subject is human.
26. The method of aspect 25, wherein the osteoprotegerin is human osteoprotegerin.
27. The method of any one of aspects 22-26, wherein multiple therapeutically effective doses of the composition are administered to the subject.
28. The method of aspect 27, wherein multiple cycles of treatment are administered to the subject for a time period sufficient to effect at least partial regeneration or repair of the bone.
29. The method of aspect 28, wherein multiple cycles of treatment are administered to the subject for a time period sufficient to effect complete regeneration or repair of the bone.
30. The method of any one of aspects 22-29, wherein the composition is administered by submucosal, intraperiosteal, or extra-bony injection.

### EXPERIMENTAL

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

All publications and patent applications cited in this specification are herein incorporated by reference as if each individual publication or patent application were specifically and individually indicated to be incorporated by reference.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### Example 1

### Local Sustained Intraoral Delivery of Anti-Osteolytic Osteoprotegerin (OPG) Via Osteoconductive Hollow Hydroxyapatite (HHAP) Microparticles

### Introduction

Here, we describe the controlled, sustained, and localized delivery of osteoprotegerin (OPG) in bioactive hydroxyapatite microparticles (HHAP) as a novel therapeutic approach to mitigate post-orthodontic relapse and for use in localized bone regenerative therapies. Given that there are no other viable approaches to predictably mitigate post-orthodontic relapse, this approach has enormous potential in the field of orthodontics. The proposed drug delivery approach would be a first of its kind, highly innovative, and could predictably improve orthodontic treatment outcomes. We envision this product will be administered by the clinician chair side just prior to removal of tooth moving appliances. Besides the application of this invention of delivering a biologic (OPG) encapsulated in a bioactive slow-release delivery system for post-orthodontic retention, the technology will also be applicable for use in a broader range of musculoskeletal applications such as alveolar bone defects in periodontal disease, post-traumatic and post-surgical bone reconstruction and other similar situations in which rapid and robust bone maturation/regeneration is required.

### Methods

### Calcium Carbonate Template Synthesis

To produce calcium carbonate hard templates, a 5 mL solution containing 0.1 M Calcium Nitrate in 0.1% Polystyrene Sulfonate is chilled on ice with continuous mixing at 500 RPM for 5-minutes. After 5-minutes of chilling, a 5 mL solution containing 0.1 M Sodium Carbonate solution in 0.1% Polystyrene Sulfonate is added dropwise (5 ml/minute) into the 0.1 M Calcium Nitrate solution to induced calcium carbonate precipitation. After dropwise addition, the calcium carbonate precipitate is aged for 1-5 minutes and monitored for crystal growth via bright field microscopy. After aging, the calcium carbonate microparticles are centrifuged at 3,000 RPM for 1 minute to pellet and the supernatant is removed. Calcium Carbonate microparticles are washed thrice with 0.1% Polystyrene sulfonate in water.

### Hollow Hydroxyapatite Synthesis

A 0.93 M Sodium Phosphate solution is prepared in water and warmed to 80° C. prior to hydrothermal conversion. Once the desired temperature is achieved, the 0.93 M solution is pH adjusted to either 9.14 (Solution A) or 11.01 (Solution B). Washed calcium carbonate microparticles are suspended in either 10 mL of Solution A or Solution B in a 100 mL Pyrex glass bottle and pipetted until a homogenous solution is attained. After, the suspended calcium carbonate microparticles are transferred to a generic autoclave to undergo hydrothermal conversion at 122° C. for 120 minutes. Post hydrothermal conversion the as-prepared hydroxyapatite coated calcium carbonate microparticles are cooled for 15 minutes on an orbital shaker at 100 RPM. After cooling, they are pelleted at 3,000 RPM and washed thrice with double distilled water. The inner calcium carbonate core is etched out with a 0.1 M Acetic Acid solution for exactly 5 minutes (including pelleting time) with constant agitation at 14 RPM. The solution is removed and the now hollow hydroxyapatite microparticles are washed with double distilled water thrice and lyophilized for 48 hours.

### Hollow Hydroxyapatite Characterization

Scanning electron microscopy (SEM) samples were prepared on carbon adhesive disc and sputter coated with pallidum/gold with a thickness of 1.3 nm. Images were obtained on a Sigma VP500 (Carl Zeiss Microscopy, and Quanta 3D FEG, FEI) at 1 keV. Transmission electron microscopy (TEM) samples were prepared by placing 5 μL of a 0.25 mg/ml hollow hydroxyapatite solution on a Formvar-coated 200-mesh copper grid and dried at 37° C. overnight. TEM imaging was performed on a JEOL JEM-1400 (JEOL USA) operating at 120 kV. Data were recorded with a 4 k Gatan Ultra Scan CCD camera (Gatan). Phase analysis was carried out using a Rigaku Miniflex 6G Benchtop Powder XRD, operated at 40 kV and 15 mA, and with a scan speed of 10°/min. Attenuated Total Reflectance Fourier Transform Infrared Microscopy (ATR FTIR) was performed with a scan resolution of 4 cm⁻¹ from 4500 to 450cm⁻¹. ATR FTIR measured were ensured to have similar amounts of powder and anvil pressure with a value of 45. A background scan was recorded immediately after each sample scan to facilitate background correction. Further analysis was down using the Spectrum software to baseline, ATR correct, and normalize the data to the highest ordinate value. The final spectra were transferred to MATLab wherein the integrated areas of the v₁v₃ phosphate (PO₄³⁻, 895-1,215 cm⁻¹) and v₂ carbonate (CO₃²⁻, 840-890 cm⁻¹) bands were calculated as previously reported¹. Carbonate-to-phosphate (CO₃²⁻/PO₄³⁻) ratios were calculated from integrated areas of the respective peaks. Additionally, peak heights were measured at specific wave numbers: 1,112, 1,020, and 1,030 cm⁻¹. From these, a series of absorbance ratios were calculated to determine additional spectroscopic parameters. The ratio of 1,030 to 1,020 cm⁻¹ represents the ratio of stoichiometric apatite to nonstoichiometric apatite, a measure of crystallinity. The ratio of 1112 to 1030 cm⁻¹ represents the ratio of fresh precipitate to phosphate, termed mineral immaturity index. Higher mineral immaturity index indicates more recently deposited mineral. For Brunauer-Emmett-Teller (BET) surface area analysis, samples were heat activated at 100° C. and then subjected to nitrogen absorption and desorption.

### Arsenazo III Calcium Detection Assay

To detect calcium release from hollow hydroxyapatite microparticles, 5 mg was suspended into a 350 μL solution containing 20 mM HEPES buffer (pH 7.4). Microparticles were pelleted at 3,000 RPM for 1 minute at predetermined timepoints and the full solution was collected and stored at 4° C. A fresh 350 μL was added after collection. A 0.4 mM Arsenazo III solution in 20 mM HEPES buffer (pH 7.4) was made and then 180 μL added to a 96-well plate. 20 μL of each sample was added to the 180 μL and the reaction was allowed to happen for 5 minutes a room temperature. Absorbance was collected on a plate reader at 620 nm.

### Protein Loading and Release Characterization

5 mg of hollow hydroxyapatite was placed in a Lo-protein binding tube and then a 350 μL solution of PBS containing 0.943 mg of OPG was added. After gentle pipetting, the tube was placed on ice and under vacuum to facilitate vacuum assisted absorption into lumen of the hollow microparticle. After loading, the hollow hydroxyapatite microparticles were pelleted and wash thrice. Each was collected and stored at 4° C. After washing, the OPG-loaded hollow hydroxyapatite microparticles were suspended in 350 μL of DPBS to assess the release profile from the microparticles. At predetermined timepoints, the microparticles were pelleted and the solution was collected and stored at 4° C. The loading efficiency and protein release amount was determined by performing a microBCA assay according to manufactures protocols.

### Mineral Layer Modifications

To induce mineral layer-by-layer precipitation, a previously reported modified simulated body buffer was utilized². The microparticles were loaded as previously mentioned and then added to 5 mL of the modified simulated body buffer solution. After 48 hours, the solution was collected and stored at 4° C. to assess the amount of OPG lost in solution. This process was repeated once more to induce 2 layers of modifications.

### Results

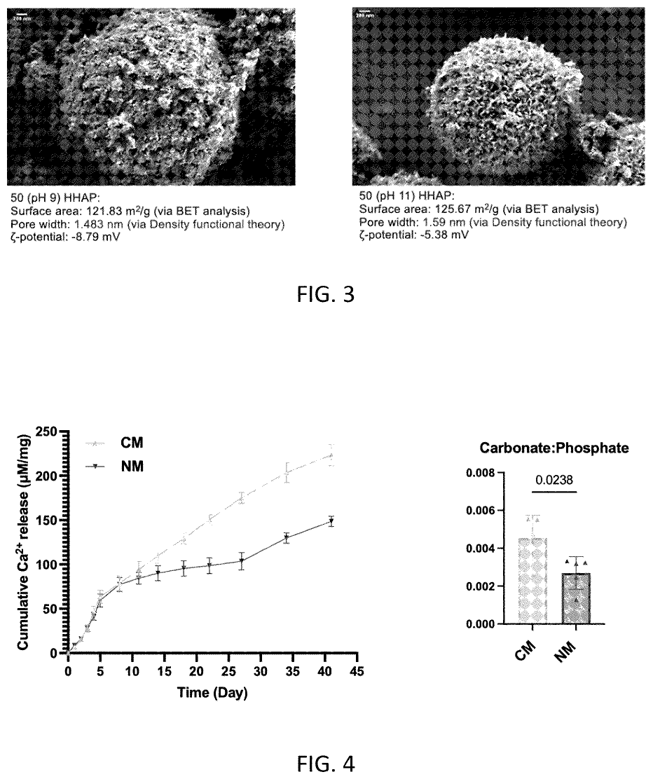

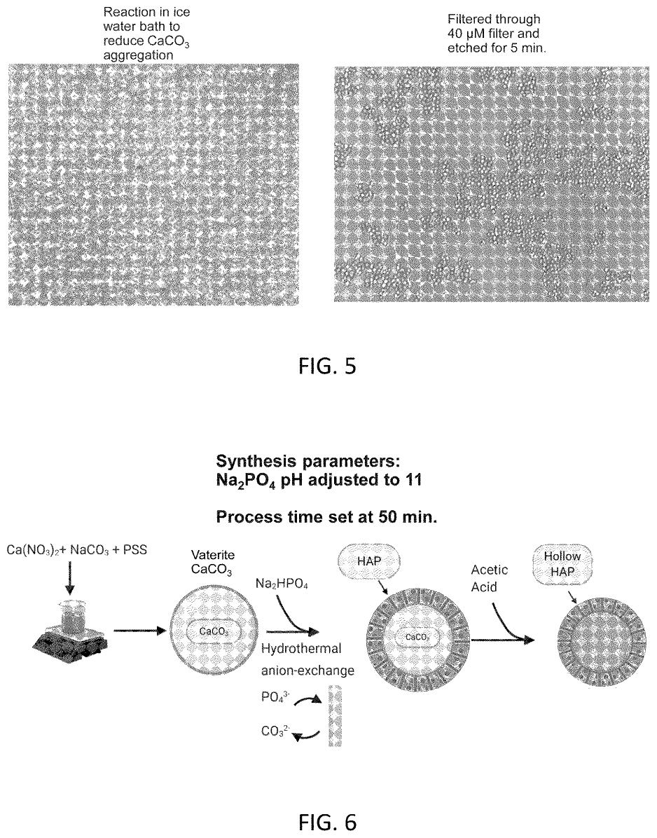

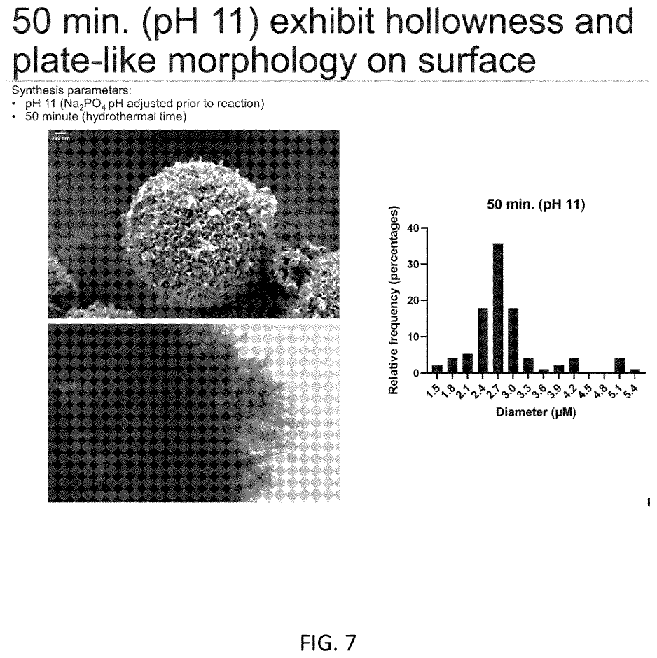

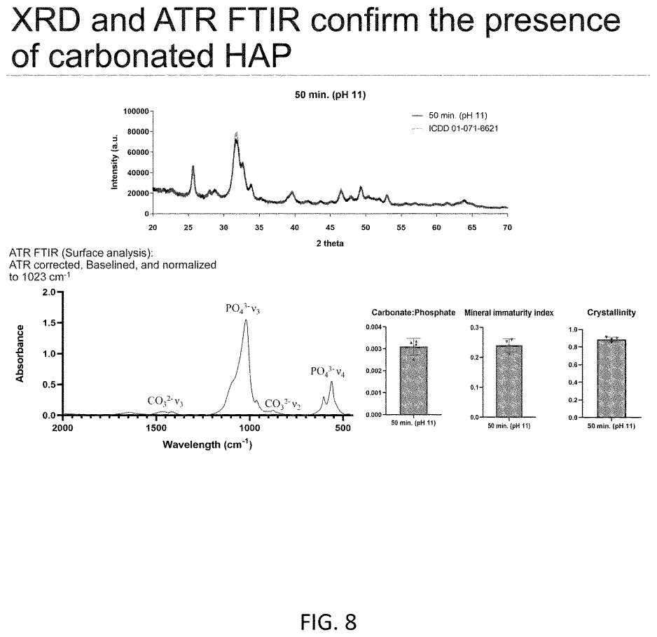

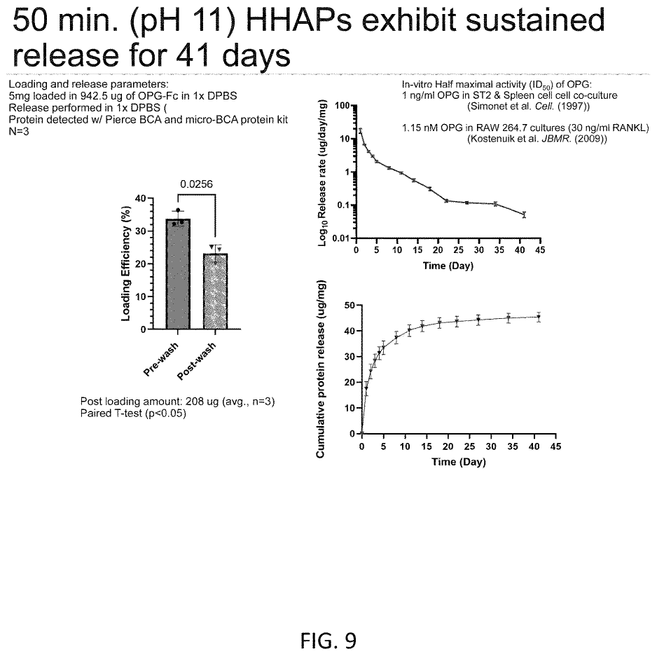

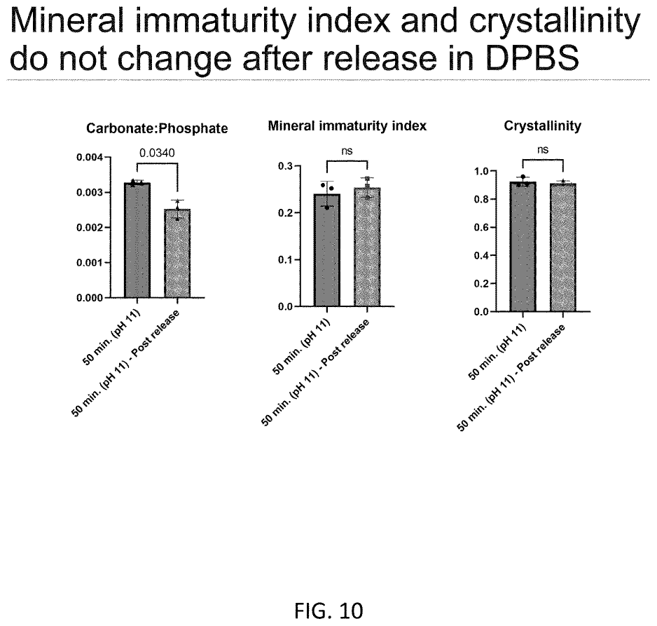

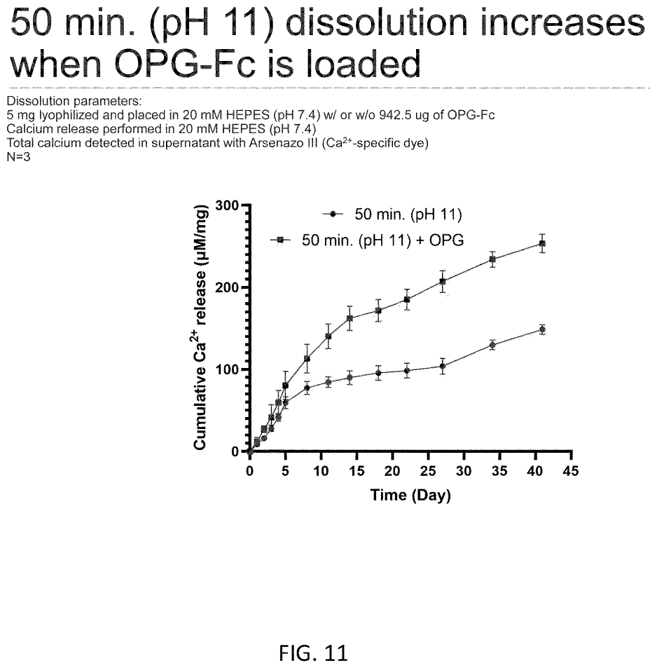

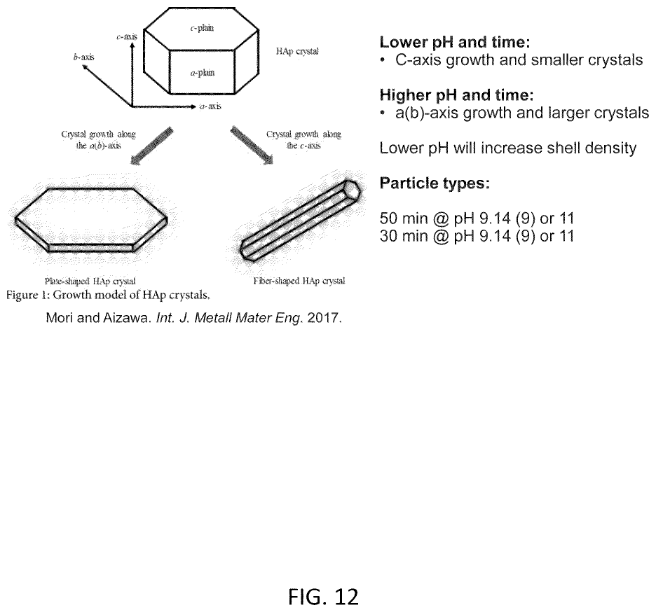

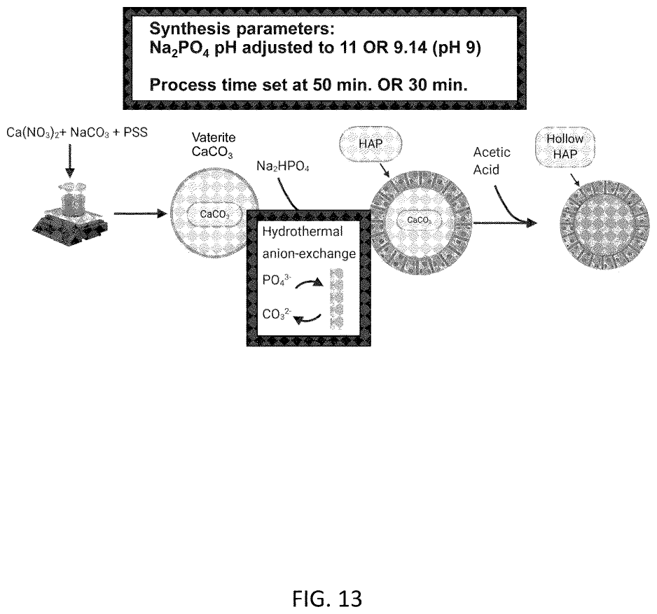

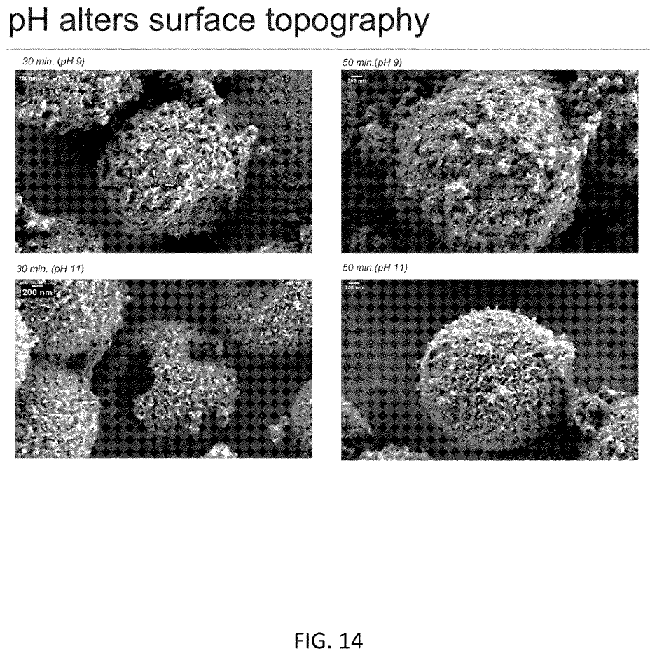

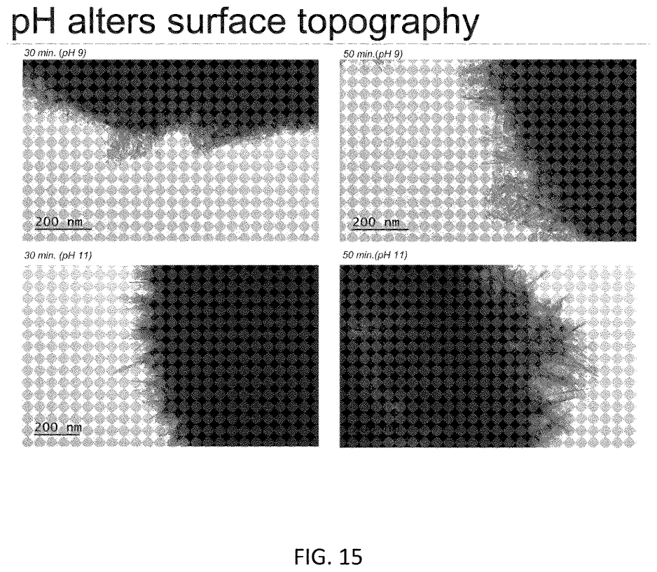

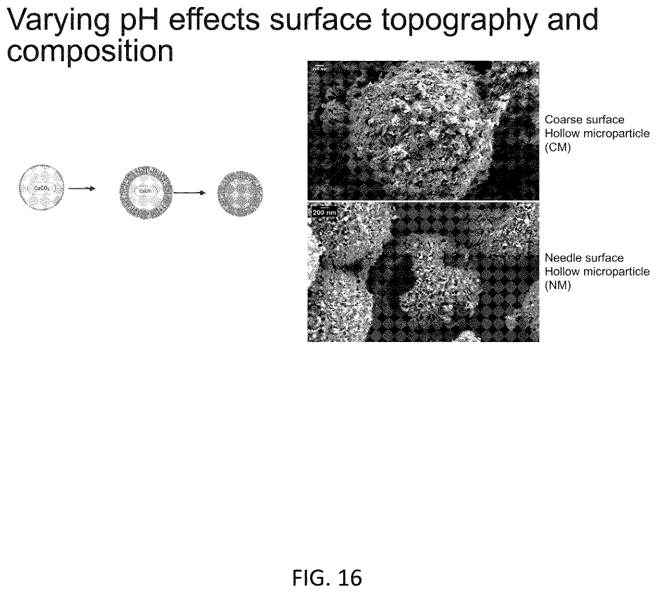

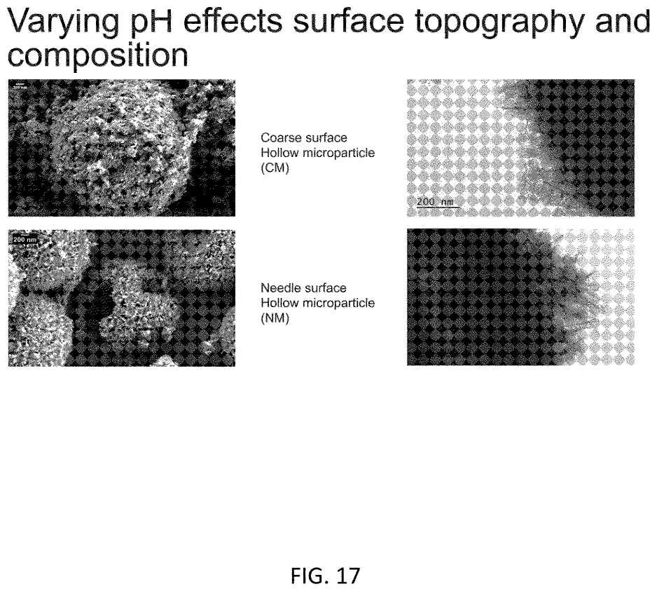

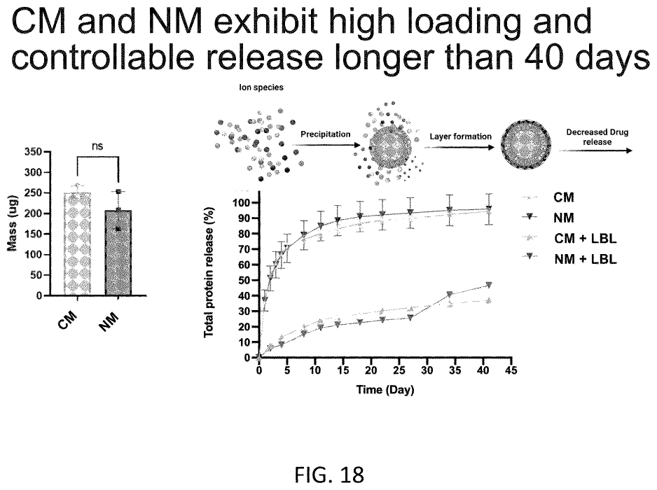

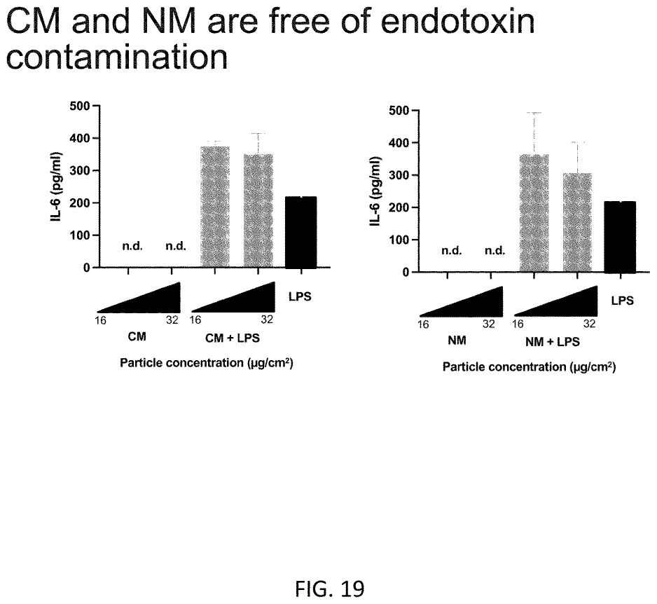

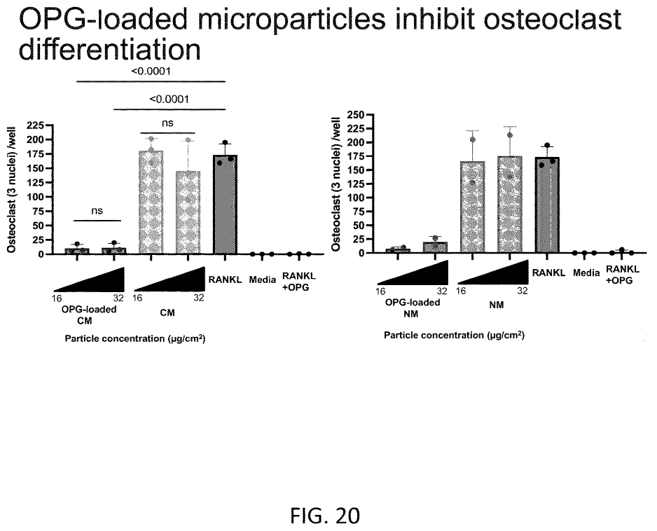

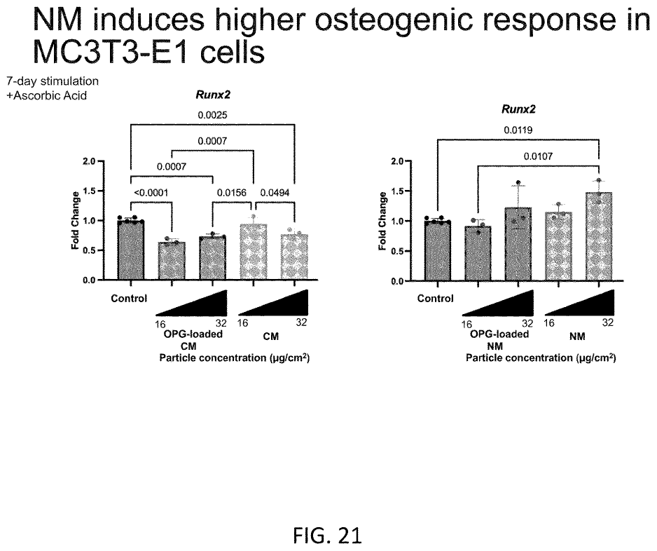

Calcium carbonate templating synthesis produces rapid formation of the vaterite phase calcium carbonate mineral in less than 5 minutes presumably due to reducing the kinetic energy in the system via the cold-water bath. The synthesis conditions for producing the hydroxyapatite shell can be further modified by changing the pH with a sodium hydroxide and hydrochloric acid solution. pH alters the surface crystal topography while maintaining the internal hollow cavity. BET analysis and density functional theory modeling indicate the pH 11 microparticles have a higher surface area and pore width, but a lower zeta potential (i.e., higher surface charge) (FIG. 3).

FTIR analysis indicates that the pH 9 hollow hydroxyapatite have a higher carbonate substitution, and this increases the calcium dissolution due to destabilizing the apatite crystal lattice (FIG. 4).

### Example 2

### Local Sustained Intraoral Delivery of Anti-Osteolytic Osteoprotegerin (OPG) Via Osteoconductive Hollow Hydroxyapatite (HHAP) Microparticles to Mitigate Post-Orthodontic Relapse

Orthodontic relapse, which entails the rebound of teeth towards their pretreatment positions, is one of the most pressing issues in the field. Due to orthodontic relapse, patients often require repeat treatment to correct their misaligned teeth to decrease the risk of future functional and dental problems including tooth decay, gum disease, and difficulties breathing and/or chewing at substantial cost to patients and reputational and medico-legal risks to providers. Currently, clinicians rely on removeable or bonded retainers to prevent teeth from shifting back to their original positions. However, patient compliance with removable retainers is unpredictable and bonded retainers suffer from long-term failure and increased calculus and plaque deposition.

We use sustained delivery of anti-osteolytic OPG with osteoconductive HHAP microparticles, which synergistically inhibit bone resorption and enhance bone maturation, respectively, to robustly mitigate post-orthodontic relapse. Our approach is the first to use biologics for post-orthodontic stabilization of teeth and mitigate or substantially minimize the need for patients to wear compliance-based removable retainers or fixed retainers that contribute to increased propensity for plaque/calculus deposition and dental disease. In addition, our method has the potential to predictably minimize relapse, which remains highly unpredictable and occurs at very high incidence with current mechanical devices used to stabilize teeth.

### Example 3

### Local Sustained Intraoral Delivery of Anti-Osteolytic Osteoprotegerin (OPG) Via Osteoconductive Hollow Hydroxyapatite (HHAP) Microparticles for Treatment of Periodontitis

Periodontitis results from chronic infection and host responses affecting ˜25-30% of the adults in the US and manifests as resorption of the bone that supports teeth. The bone loss accompanying periodontitis leads to teeth becoming loose, falling out, or that have to be extracted by a clinician. Currently, while the infection causing periodontitis can be treated with scaling/root planning with or without antibiotics, there are limited therapies to treat and recover the bone loss associated with the disease. Most patients with moderate to severe bone loss undergo surgical interventions and/or regenerative therapies. As with the case of bone defects and fractures, periodontitis bone loss is currently treated with bone grafts with similar associated disadvantages seen in the case of bone fractures/defects. Other bone regenerative therapies for periodontal bone loss include allogeneic demineralized bone matrix with variable success rates and potential for graft-host response. As such, there is an unmet need for new biologics and delivery systems to repair localized bone loss of patients with periodontitis-associated bone loss.

We use sustained delivery of anti-osteolytic OPG with osteoconductive HHAP microparticles to treat periodontitis-associated bone loss.

### Example 4

### Local Sustained Delivery of Anti-Osteolytic Osteoprotegerin (OPG) Via Osteoconductive Hollow Hydroxyapatite (HHAP) Microparticles for Bone Regenerative Therapies

Bone defect repair is one of the most common regenerative procedures with more than 2 million bone grafts performed worldwide annually. The major cause of large bone defects is trauma, congenital anomalies, and pathologic bone loss including that due to periodontal disease and following tissue resection due to cancer. Although bone typically has the capacity to fully regenerate, it has limited ability to bridge very large defects. Additionally, the robustness and rate of regeneration varies significantly making the outcomes unpredictable. Below is a summary of some of the unmet needs of two specific situations in bone regenerative therapies where treatment with OPG delivered with HHAP microparticles offers distinct advantages over current approaches for bone regeneration.

In the U.S., ˜15 million bone fractures occur each year. Approximately 5-10% of these cases exhibit delayed healing or non-union of the fractured bone. Delayed healing increases to almost 50% in patients with vascular damage or co-morbidities such as diabetes, old age, smoking, and obesity. Current standards of care utilize surgical interventions with autologous bone grafts to increase stability or promote healing. However, autologous bone grafts are prone to donor site morbidity, bone adsorption, and infection. There are currently no pharmacological agents approved to accelerate or treat nonunion bone fractures. Bone Morphogenetic Proteins (BMPs), specifically BMP-2, is the only FDA approved biologic for problematic fractures with a very narrow indication window associated with surgical implantation within a carrier. However, clinical application of BMP-2 has become limited due to its high cost and growing list of adverse outcomes. As such, there remains an unmet need for alternative biologics and carrier systems that stimulate fracture healing. OPG can be delivered via osteoconductive HHAP microparticles to promote a robust and expedited bone repair in fractures and non-unions. The likely patient who will benefit from such treatment include individuals with increased susceptibility to fractures such as elderly patients or patients with comorbidities.

For bone regeneration, the gold standard of care requires autologous bone harvested from a donor site. However, harvesting bone from the donor site has significant limitations including donor site morbidity, infection, and potential damage to the surrounding blood vessels and nerves. Our invention overcomes these limitations such that all the materials are made without harvesting anything from the donor. Using OPG delivered via osteoconductive HHAP microparticles overcomes the limitations of allogenic grafts and demineralized bone matrix of host-graft responses. Current biologics used for bone regeneration includes BMP-2. However, BMP-2 is associated with high cost and has complex side effects such as ectopic bone formation, higher bone resorption, and tumorigenesis. OPG has been used in phase I clinical trials and did not have any severe adverse outcomes. Additionally, HAP has been approved by the FDA as a bone filler. The combination therapy of HAP/OPG overcomes the limitations associated with BMP-2.

### Example 5

### In-Vitro Osteoclast Assays

HHAP microparticles are incubated in RANKL-media at pre-determined timepoints with osteoclasts. For comparison, osteoclasts are incubated with OPG-loaded HHAP microparticles, empty HHAP microparticles, soluble OPG-Fc, or HHAP+OPG.

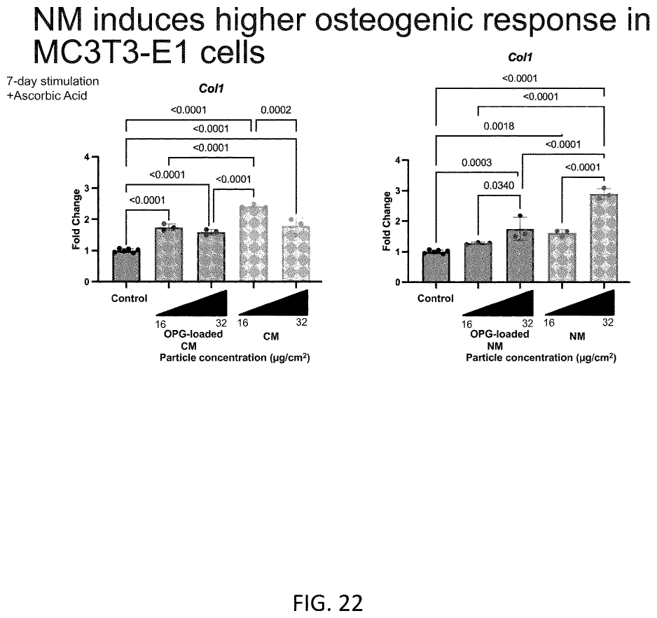

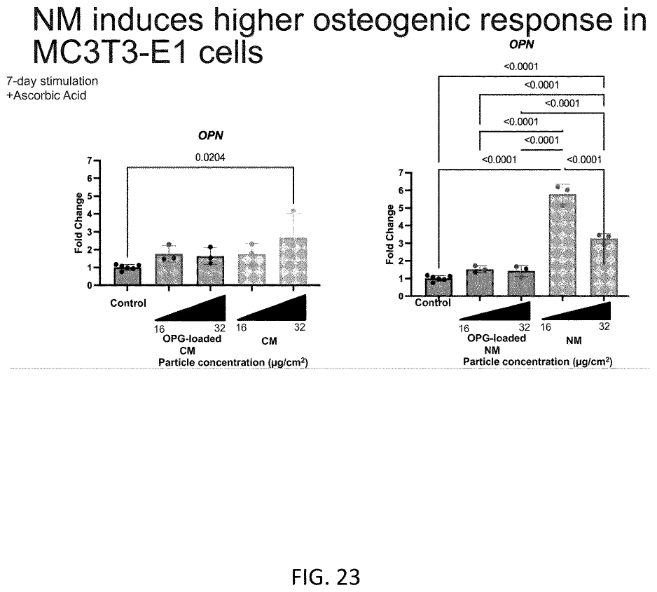

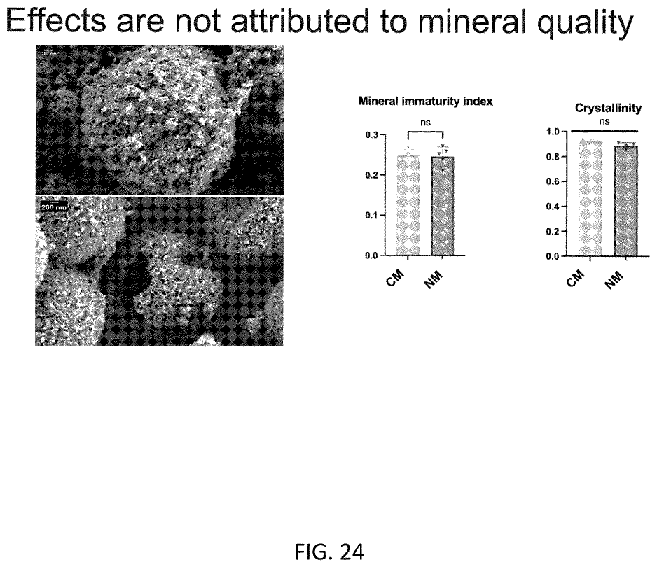

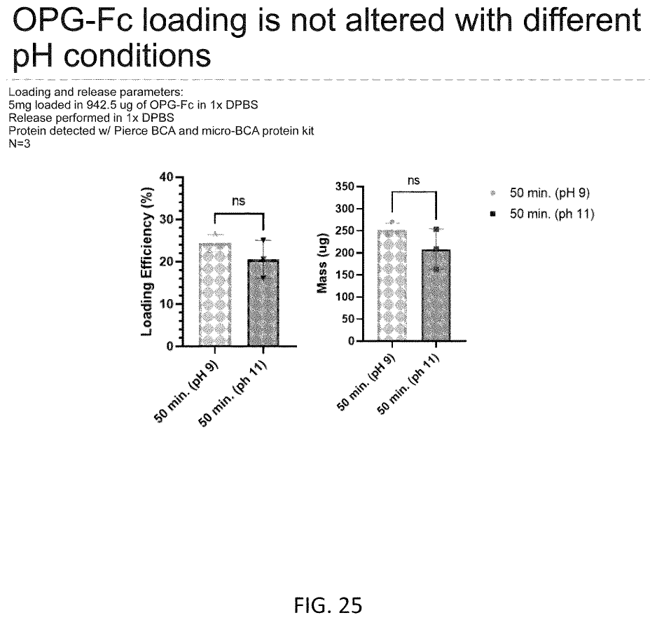

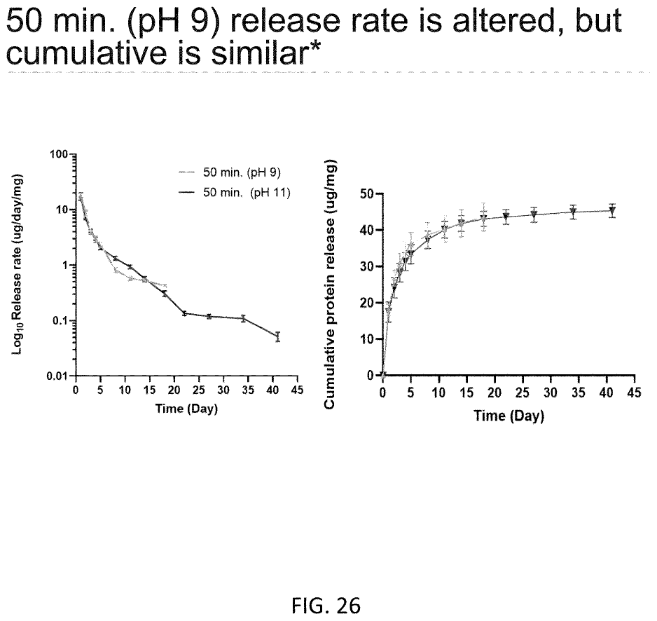

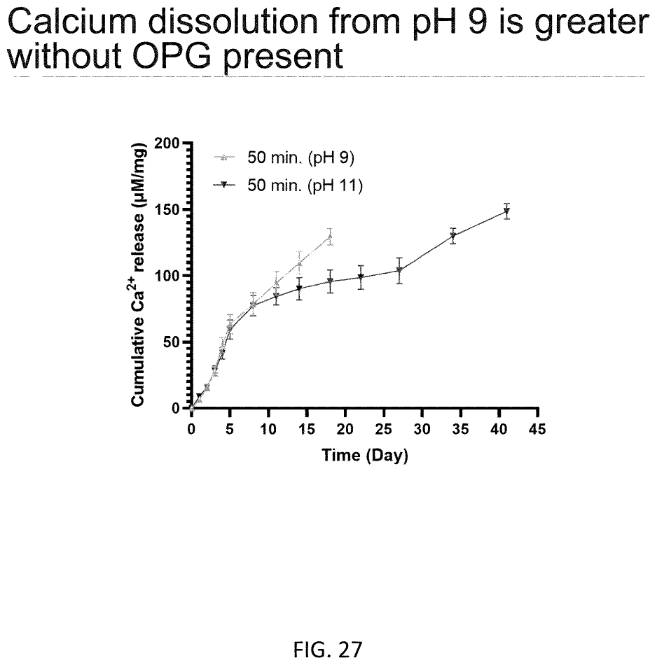

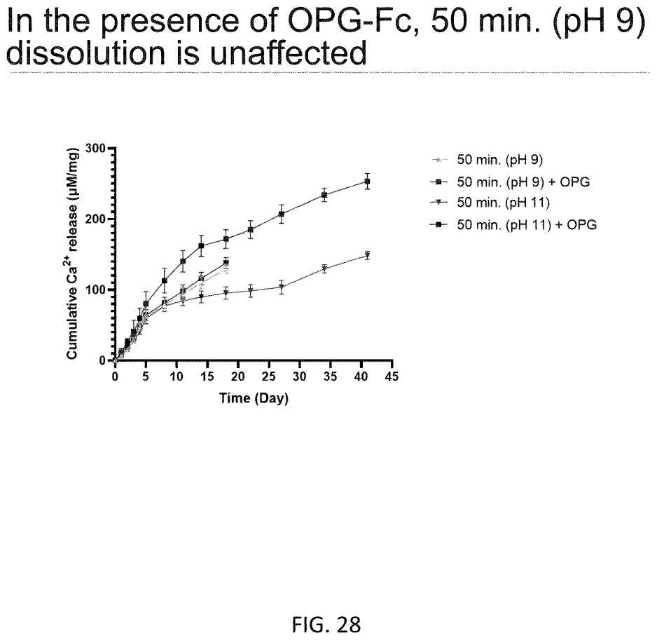

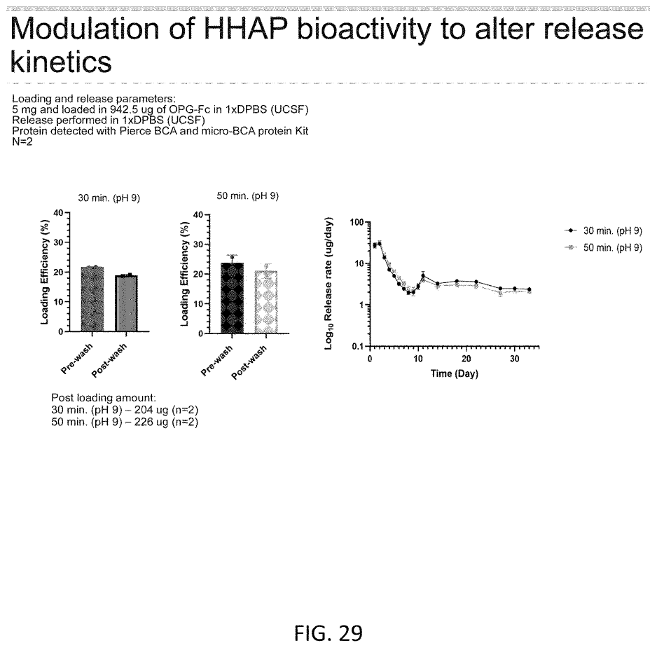

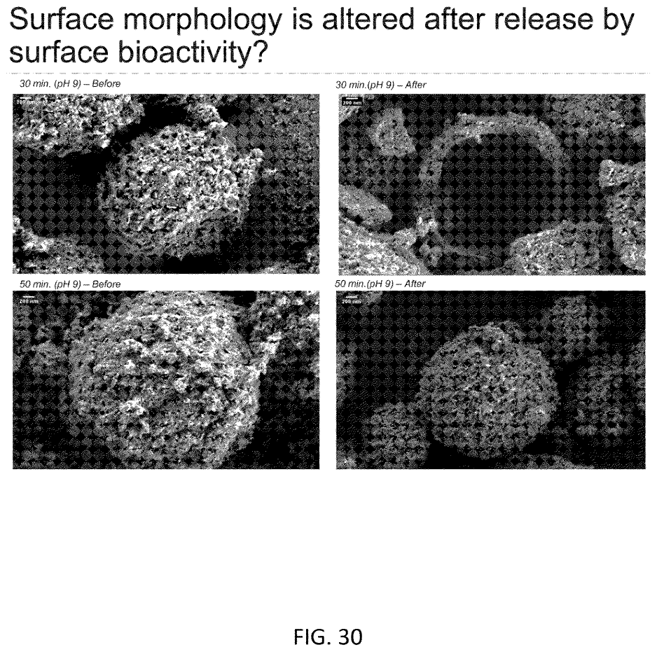

The effects of the OPG-loaded HHAP microparticles on osteoblast differentiation were assayed by detecting gene expression of Col1, Runx2, and Ocn in MC3T3-E1 cells. As shown in FIG. 21, OPG-loaded microparticles inhibited osteoclast differentiation. NM microparticles induced higher osteogenic responses in MC3T3-E1 cells that CM microparticles (FIGS. 22-24).

What is claimed is:

1. A microparticle comprising:
a) a hollow core;
b) a layer of hydroxyapatite encapsulating the hollow core; and
c) an agent that provides osteoprotegerin anti-osteolytic activity for bone regeneration or post-orthodontic tooth stabilization, wherein the agent that provides osteoprotegerin is contained within the hollow core.

2. The microparticle of claim 1, wherein the agent that provides osteoprotegerin anti-osteolytic activity is osteoprotegerin.

3. The microparticle of claim 1, wherein the agent that provides osteoprotegerin anti-osteolytic activity is a fusion protein comprising osteoprotegerin.

4. The microparticle of claim 3, wherein the fusion protein comprises osteoprotegerin linked to an immunoglobulin constant fragment (Fc).

5. The microparticle of any one of claims 1-4, wherein the osteoprotegerin is human osteoprotegerin.

6. The microparticle of any one of claims 1-5, wherein the microparticle is a coarse surface hollow microparticle (CM) or a needle surface hollow microparticle (NM).

7. The microparticle of any one of claims 1-6, wherein the microparticle has a diameter ranging from about 1.5 μm to about 5.4 μm as measured by transmission electron microscopy (TEM).

8. The microparticle of claim 7 wherein the microparticle has a diameter of 2.7 μm±0.3 μm as measured by transmission electron microscopy (TEM).

9. The microparticle of any one of claims 1-8, wherein the layer comprising hydroxyapatite has a thickness sufficient to allow sustained release of effective amounts of the OPG from the microparticle for at least 40 days.

10. A composition comprising the microparticle of any one of claims 1-9 and a pharmaceutically acceptable excipient or carrier.

11. The composition of claim 10, wherein the composition comprises an effective dose of the agent that provides osteoprotegerin anti-osteolytic activity to stimulate bone formation and decrease bone resorption.

12. A composition comprising the microparticle of any one of claims 1-9 for use in a method of treating post-orthodontic relapse.

13. A composition comprising the microparticle of any one of claims 1-9 for use in a method of treating a bone disorder.

14. The composition of any one of claims 10-13, wherein the carrier is selected from the group consisting of an aqueous solution, a gel, a lotion, a balm, or a paste.

15. A kit comprising the composition of any one of claims 10-14 and instructions for using the composition for treating molar relapse or regenerating bone for treatment of a bone disorder.

16. A method of treating post-orthodontic relapse in a subject in need thereof, the method comprising administering a therapeutically effective amount of the composition of any one of claims 10-14 to the subject before, after, or during an orthodontic procedure to mitigate or prevent post-orthodontic relapse.

17. The method of claim 16, wherein the composition is administered submucosally or topically.

18. The method of claim 16, wherein the composition is administered by intrasulcular or peri-radicular micro-injection.

19. A method of treating periodontal disease in a subject in need thereof, the method comprising administering a therapeutically effective amount of the composition of any one of claims 10-14 to the subject.

20. The method of claim 19, wherein the composition is administered submucosally or topically.

21. The method of claim 19, wherein the composition is administered by intrasulcular or peri-radicular micro-injection.

22. A method of treating a subject having a bone in need of bone regeneration or repair, the method comprising locally administering to the bone a therapeutically effective amount of the composition of any one of claims 10-14.

23. The method of claim 22, wherein the bone has a defect caused by trauma, surgery, a congenital anomaly, or pathological bone loss.

24. The method of claim 23, wherein the pathological bone loss is caused by cancer, periodontal disease, osteoporosis, arthritis, or osteonecrosis.

25. The method of any one of claims 22-24, wherein the subject is human.

26. The method of claim 25, wherein the osteoprotegerin is human osteoprotegerin.

27. The method of any one of claims 22-26, wherein multiple therapeutically effective doses of the composition are administered to the subject.

28. The method of claim 27, wherein multiple cycles of treatment are administered to the subject for a time period sufficient to effect at least partial regeneration or repair of the bone.

29. The method of claim 28, wherein multiple cycles of treatment are administered to the subject for a time period sufficient to effect complete regeneration or repair of the bone.

30. The method of any one of claims 22-29, wherein the composition is administered by submucosal, intraperiosteal, or extra-bony injection.
